(19) United States
(12) Patent Application Publication
(10) Pub. No.: US 2022/0322770 A1
(43) Pub. Date: Oct. 13, 2022

(54) Soft Elastic Disposable Gloves

(71) Applicant: Xiaoqing Deng, Lincolnshire, IL (US)

(72) Inventor: Xiaoqing Deng, Lincolnshire, IL (US)

(21) Appl. No.: 17/227,293

(22) Filed: Apr. 10, 2021

(51) Int. Cl. A41D 19/00; A41D 31/18

(52) U.S. Cl. CPC A41D 19/0006; A41D 31/185

(57) ABSTRACT

A single use disposable glove utilizing the cutting and sealing process. The top and bottom panels of the glove are produced from an extrusion process made from a variety of thermoplastic elastomers containing styrenic block copolymer (SBC). The glove made from this process is form fitting, has less than 20% deformation after 100% stretch for 10 second. The suitable SBC materials are selected from the group consisting of SIS, SBS, SEBS, SEPS, SEEPS, and combinations thereof.

### FIELD OF THE INVENTION

The present invention generally relates to disposable gloves made by welding two flat panels (or films) made from styrenic block copolymer (SBC) based thermoplastic elastomer materials.

### BACKGROUND OF THE INVENTION

Single use disposable gloves have been produced for the purposes of protection and hygiene including, but not limited to medical procedures and examinations, as well as for use in the food service industry. These types of disposable gloves are employed to protect both the user and other individuals from contact with various germs or pathogens. Generally, these types of disposable gloves are manufactured using a dipping method or a cutting and sealing method.

The dipping method would employ a three-dimensional hand shaped former (also called a mold) which is introduced into a forming liquid compound, such as a natural or synthetic latex. A portion of the forming liquid compound would adhere to the hand shaped former to produce a thin layer of film thereon. After this thin layer of film solidifies, the thin layer film would be stripped from the former, thereby producing a glove.

The dipping processes as described above can produce elastomeric articles, such as gloves, that are elastic, are form-fitting, are soft and pliable, have barrier protection, and have tactile sensitivity. Unfortunately, however, the above-described dipping process is both labor and energy intensive. Further, only certain types of elastomeric polymer materials are amenable to the dipping process. It is also very difficult (if not impossible) to manufacture gloves less than 0.05 mm thickness while maintaining all features using the dipping process. In practice, most medical gloves are made using the dipping process. Another key disadvantage of dipped gloves is that the dipping process is not environmentally friendly as it has to rely on solvent to dissolve the polymers and these solvents are volatile organic compounds. As the end product is a chemically crosslinked rubber, it is not recyclable.

In an alternative manufacturing process, instead of producing disposable gloves through a dipping process, gloves can also be produced by welding two flat panels. This process is also called the “cutting and sealing” method. Forming a disposable glove through a welding process can be much less expensive than the dipping process. Unfortunately, problems have been experienced in the past to produce welded gloves that have desired protective properties comparable to gloves made from the dipping process. In this regard, the welded gloves typically do not have form-fitting properties, have poor barrier protections, and are oversized in relation to a hand resulting in a poor fit. In practice, most disposable gloves used in food service have been made from welding two flat polyethylene (PE) films.

Gloves made by welding two flat films can have much smaller thickness (<0.05 mm), which helps drive unit cost down in addition to low costs of raw materials. Gloves made from the dipping processes tend to have superior properties, especially deformation after 100% stretch (see Table 1) compared to gloves made by welding two flat PE panels. For example, gloves made by the dipping process using PVC, NRL or Nitrile can often have less than 16% or even less than 10% deformation after 100% stretch, while gloves made by welding two flat PE panels have >30% deformation after 100% stretch.

As shown in Table 1, PE gloves made using welding process has inferior properties such as high deformation, compared to Nitrile gloves made using the dipping process. Although PE gloves can be made much thinner, the deformation after 100% stretch has been tripled, indicating that they are easier to lose the original shape and have low puncture resistance. In the market today, there are no welded gloves have been manufactured using the cost-effective welding process while maintaining properties comparable to dipped Nitrile gloves.

In view of the above, there is a need for producing welded gloves with similar or superior properties compared to dipped nitrile gloves.

### SUMMARY OF THE INVENTION

In general, the present disclosure is directed to a disposable glove, made from two flat thermoplastic elastomer panels. In one embodiment, each panel comprises a single layer film. In another embodiment, each panel comprises a multi-layer film. The multi-layer film may comprise, for instance, a co-extruded film. Once the panels are formed, the panels are then used to produce elastic articles, such as gloves, by welding the panels together. For example, a first hand-shaped panel may be welded to a second hand-shaped panel about their peripheries in order to form a disposable glove. The second panel may or may not be the same as the first panel. The first and second panels may be made from different materials or may have different thicknesses.

In the manner described above, gloves can be produced without having to dip a mold into a dipping solution in order to form the article.

In one embodiment, for instance, the present disclosure is directed to a glove having form-fitting properties when worn by a user. The glove includes a first hand-shaped panel welded to a second hand-shaped panel about their peripheries or outer perimeters. The two panels are welded together leaving a hollow opening for receiving a hand. In accordance with the present disclosure, each of the two hand-shaped panels comprises a Styrenic Block Copolymer based thermoplastic elastomeric film. The film may be made from a mixture of an SBC and other ingredients such as polyethylene (PE). The SBC may be selected from the group consisting of SIS, SBS, SEBS, SEPS, and SEEPS. In one embodiment, the SBC contains 12% to 25% styrene by weight in the SBC. Having styrene content within this range was found to be advantageous in terms of the ability to make thin films with a uniform thickness, besides the favorable deformation after 100% stretch for 10 seconds. With Styrene content in this range, the formed glove is soft and comfortable to wear.

Other features and aspects of the present disclosure are discussed in greater detail below.

### BRIEF DESCRIPTION OF THE DRAWINGS

The above and additional objects, characteristics, and advantages of the present invention will become apparent in the following detailed description of preferred embodiments, with reference to the accompanying drawings.

FIG. 1 is a top view of two welded thin film layers of a thermoplastic elastomer material.

FIG. 2 is a side view of two welded thin film layers of a thermoplastic elastomer material.

FIG. 3 is a broad view of a thermoplastic elastomer glove.

Identical reference numerals throughout the figures identify common elements.

### DESCRIPTION OF THE PREFERRED EMBODIMENTS

### Definitions

As used herein, “PVC” means polyvinyl chloride; “NRL” means natural rubber latex; “PE” means polyethylene; “TPE” means thermoplastic elastomer.

As used herein, and consistent with ISO 1382, “thermoplastic elastomer” is a polymer or a mixture of polymers which doesn't need vulcanization or reticulation when processed, but demonstrates similar properties as vulcanized rubber at service temperature. These properties disappear at processing temperature to allow the ulterior process possible, but reappear as the material returns to working temperature.

As used herein, the term “elastomeric” and “elastic” refer to a material that, upon application of a stretching force, is stretchable in at least one direction, and which upon release of the stretching force, contracts/returns to approximately its original dimension. For example, a stretched material may have a stretched length that is at least 50% greater than its relaxed unstretched length, and which will recover to within at least 50% of its stretched length upon release of the stretching force. A hypothetical example would be a one (1) inch sample of a material that is stretchable to at least 1.50 inches and which, upon release of the stretching force, will recover to a length of not more than 1.25 inches. Desirably, the material contracts or recovers at least 50%, and even more desirably, at least 80% of the stretched length, and most desirably, at least 90% of the stretched length.

As used herein, “SBC” means Styrenic Block Copolymer. As the name implies, a SBC is made of blocks of distinct polymers that each contribute to the properties of the polymer. The polystyrene end-blocks are hard polymers that add strength to the SBC elastomer. The mid-blocks are composed of rubbery polymers such as polyisoprene, polybutadiene, ethylene-butylene copolymer and others.

As used herein, “SIS” means an SBC having the structure of styrene-Isoprene-styrene;

“SBS” means an SBC having the structure of styrene-butadiene-styrene;

“SEBS” means an SBC having the structure of styrene-ethylene butylene-styrene;

“SEPS” means an SBC having the structure of styrene-ethylene Propylene-styrene;

“SEEPS” means an SBC having the structure of styrene-ethylene ethylene propylene-styrene.

As used herein, the term “weld” refers to securing at least a portion of a first polymer film with a portion of at least a second polymer film by temporarily rendering at least a portion of one film or an intermediate material into a softened or plastic state and joining the films without the use of mechanical attachments such as, for instance, stitching or without the use of an adhesive material that causes the films to stick together. Two or more films can be welded together in various ways such as through thermal bonding, ultrasonic bonding, pressure bonding, solvent bonding, or mixtures thereof.

As used herein, an “elastomer” refers to any polymer material that is elastomeric or elastic and includes plastomers.

As used herein, the tensile properties of a film including modulus and load at break are measured according to ASTM Test D412 using Die D or ASTM D-882.

As used herein, a panel that is formed from a “different composition of materials” in relation to another panel refers to any formulation variation between the two panels. Two panels having a different composition of materials may include panels made from different thermoplastic elastomers (for example one from SIS and the other from SEBS) or may include panels containing different additives or the same additives in different amounts (for example one panel is made from SIS with an oil, the other panel is made from SIS with a PE and without oil). Two panels being made from a different composition of materials can also occur when the panels contain different coloring compounds.

As used herein, a film or panel is said to be “based” on an SBC elastomer when the film or panel is made from said SBC elastomer in its pure form, or is made from a mixture of said SBC elastomer and at least one more other ingredients, including but not limited to an oil, a PE, a coloring compound or other polymers.

The present invention overcomes the poor plastic deformation after stress produced by the prior art cutting and sealing process employing polyethylene (PE) to produce the two panels of the glove. This is accomplished by utilizing a family of SBC selected from the group consisting of SIS, SBS, SEBS, SEPS, SEEPS and combinations thereof.

The use of these compositions for the top or bottom panel would produce a panel having a thickness in the range of 0.02 mm to 0.1 mm with excellent film integrity, but also good elasticity, thereby reducing hand fatigue. With improved elasticity not only would the glove be classified as truly form fitting, but it would also exhibit improved durability.

Compared to the dipping process, the cutting and sealing process employing the films produced by the above-listed compositions, would produce better film quality and reduced thickness and provide for a more versatile film structure, as well as wider glove material selection. For example, utilizing polyvinyl chloride to make a plastisol compound suitable for the manufacture of a glove using the dipping process, very limited choices of plasticizers are available. This is true, for example, since the dipping compound must be liquid at room temperature. More critically, the material that is utilized must have a viscosity at a certain range for thickness and film tensile strength optimization. Using the cutting and sealing approach, the film forming extrusion process could be produced but not limited to blowing, casting or calendaring.

Biaxial orientation is a process whereby a plastic film or sheet is stretched in such a way that the polymeric chains are oriented parallel to the plane of the film in two directions. Biaxially oriented films exhibit exceptional clarity, very high tensile properties, improved flexibility and toughness, improved barrier properties, and can be relatively easily made shrinkable.

It is our finding that the TPE films made for high elasticity and low deformation are very anisotropic and properties such as tensile strength and elongation in machine direction and transverse direction are much different. We found that through biaxial orientation, the properties, such as tensile and tear strength are improved overall.

### DESCRIPTIONS OF FIGURES

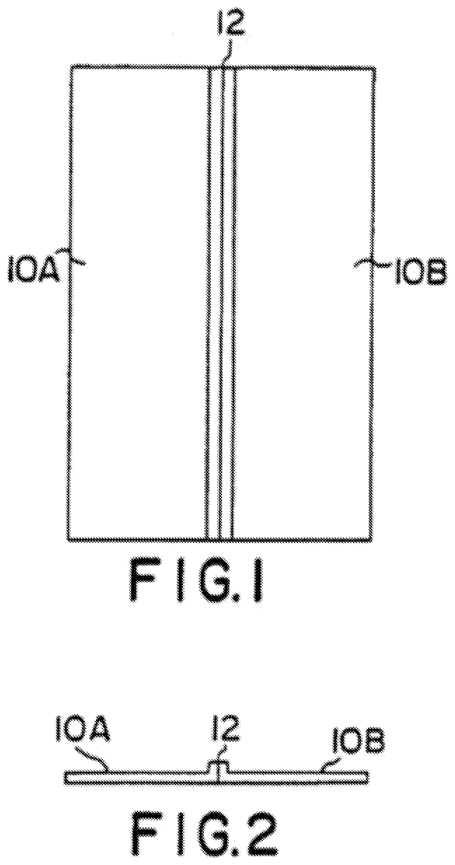

FIG. 1 is a view of panels 10A and 10B welded together to form a weld seam 12. In preparation for the welding process, a die platen is preheated prior to cutting and sealing panel 10A and panel 10B together.

FIG. 2 is a side view of joined panels (10A and 10B) of FIG. 1 with weld seam (12). FIG. 2 shows eversion of film layers (10A) and (10B) at weld seam (12). Weld seam (12) has substantially complete continuity and integrity and is without pinholes, holidays or charred edges.

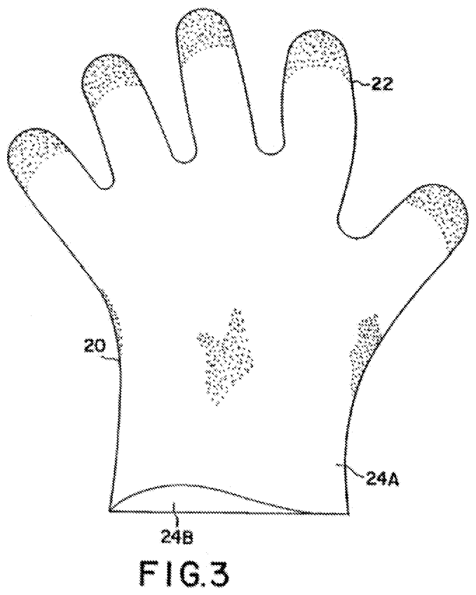

FIG. 3 is a TPE glove (20) comprised of TPE panels (top panel 24A and bottom panel 24B) joined at weld seam (22). Weld seam (22) is located at the finger contours of glove (20) where the two film layers are joined. The preheated die platen takes on the shape of the TPE product to be created, in this case, the shape of a hand. The top panel 24A is placed on top of the bottom panel 24B. A preheated die platen is lowered from above and brought into contact with these panels under pressure at the welding peripheries to create weld seam (22). The preheated die platen could take the shape of either a left hand or a right hand. Since the glove can be flipped over after the cutting and sealing process, it is necessary to define which panel is the top panel and which one is the bottom panel. For the purpose of the claim interpretation, “top panel” and “bottom panel” are defined according to their relative locations (top vs bottom) during the cutting and sealing process.

### EXAMPLES

The following examples would illustrate the present invention as compared to a prior art glove using polyvinyl chloride (PVC) in the dipping process as well as the prior art glove produced by polyethylene (PE) using the cutting and sealing process.

### Example 1

The top and bottom panels are made from the same single layer film. In this example, the film is made from a TPE comprising an SIS and a PE. The SIS has a styrene content of 18% by weight.

It is clear from Table 3 that the performance of this TPE as a glove material is excellent. It has equal or better elasticity than the industrial standard Nitrile glove material. The glove made from this TPE is very soft with a Shore A hardness of 59 and is very comfortable to wear. As it is a thermoplastic material, it can be recycled and re-used. In fact, the dropoff material from cutting the hand shaped panels is reused in making the film. The process in making the film and glove does not generate any toxic material, thus making it a truly environment-friendly process. The PE in this example may be a low density polyethylene (LDPE), a linear low density polyethylene (LLDPE), a Medium density polyethylene (MDPE), or a High density polyethylene (HDPE).

### Example 2

The top and bottom panels are made from the same single layer film based on SEBS. The SEBS has 12% to 25% (inclusive) styrene content by weight in the SBC molecular structure of the elastomer. Note again the deformation after 100% stretch is less than 10%.

### Example 3

The top and bottom panels are made from the same single layer film based on SIS and SEBS. It is found that mixing SIS and SEBS gives good balance of softness, recovery and tensile strength resulting a comfortable and strong form fitting glove.

To produce gloves with highly desired properties, the Melt Flow Indices of the top and bottom panels are preferably from 0.5 g/10 mins to 10 g/10 mins according to ASTM Test D1238 at 190 C and at a load of 2.16 kg, and the hardness of the panels is preferably in the range of 35 Shore A to 90 Shore A, and most preferably in the range of 50 Shore A to 80 Shore A.

### Example 4

The top and bottom panels are made from the same single layer film based on SBS and SEBS. In particular, the film is made from a mixture containing at least one SBS and at least one SEBS. The SBS is the lowest cost SBC and we found its use together with SEBS is synergistic and advantageous in cost control and performance.

The foregoing description, for purposes of explanation, used specific nomenclature to provide a thorough understanding of the invention. However, it will be apparent to one skilled in the art that the specific details are not required in order to practice the invention. In other instances, well known devices are shown in block diagram form in order to avoid unnecessary distraction from the underlying invention. Thus, the foregoing descriptions of specific embodiments of the present invention are presented for purposes of illustration and description. They are not intended to be exhaustive or to limit the invention to the precise forms disclosed. Obviously many modifications and variations are possible in view of the above teachings. The embodiments were chosen and described in order to best explain the principles of the invention and its practical applications, the thereby enable others skilled in the art to best utilize the invention and various embodiments with various modifications as are suited to the particular use contemplated. It is intended that the scope of the invention be defined by the following claims and their equivalents.

### Tables

TABLE 1
Typical Properties of PVC, NRL, Nitrile and PE Gloves

| Material | PVC | NRL | Nitrile | PE |
|---|---|---|---|---|
| Manufacturing Method | Dipping | Dipping | Dipping | Welding |
| Tensile Strength | 11-15 | 18-25 | 20-40 | 11-15 |
| (MPA) | | | | |
| Elongation (%) | 300-400 | ~800 | ~600 | ~600 |
| Deformation after | 8-16 | ~5 | 5-10 | >30 |
| 100% stretch (%) | | | | |
| Thickness range (mm) | 0.06-0.1 | 0.08-0.12 | 0.06-0.12 | 0.01-0.02 |
| Weight (gram) | >5 | >5 | >4 | <2 |

TABLE 2
Properties of TPE of Example 1

| Properties | Unit | Value | Test Method |
|---|---|---|---|
| Hardness | Shore A | 59 | 10 Sec; ASTM 2240 |
| Tensile Strength | MPa | 18.3 | ASTM D882 |
| Elongation | % | 852 | ASTM D882 |
| Deformation after | % | 2.5 | Per U.S. Pat. No. |
| 100% stretch | | | 8,572,765 |
| Thickness | mm | 0.05 | |

TABLE 3
Properties of TPE of Example 2

| Properties | Unit | Value | Test Method |
|---|---|---|---|
| Hardness | Shore A | 78 | 10 Sec; ASTM 2240 |
| Tensile Strength | MPa | 26.5 | ASTM D882 |
| Elongation | % | 556 | ASTM D882 |
| Deformation after | % | 8.8 | Per U.S. Pat. No. |
| 100% stretch | | | 8,572,765 |
| Thickness | mm | 0.06 | |

TABLE 4
Properties of TPE of Example 3

| Properties | Unit | Value | Test Method |
|---|---|---|---|
| Hardness | Shore A | 76 | 10 Sec; ASTM 2240 |
| Tensile Strength | MPa | 22.4 | ASTM D882 |
| Elongation | % | 778 | ASTM D882 |
| Deformation after | % | 6.3 | Per U.S. Pat. No. |
| 100% stretch | | | 8,572,765 |
| Thickness | mm | 0.06 | |

TABLE 5
Properties of TPE of Example 4

| Properties | Unit | Value | Test Method |
|---|---|---|---|
| Hardness | Shore A | 56 | 10 Sec; ASTM 2240 |
| Tensile Strength | MPa | 15.0 | ASTM D882 |
| Tensile Elongation | % | 920 | ASTM D882 |
| Deformation after | % | 9.0 | Per U.S. Pat. No. |
| 100% stretch | | | 8,572,765 |
| Thickness | mm | 0.08 | |

What is claimed is:

1. A disposable glove comprising:
one top panel in the form of a hand comprising at least one layer made from a first SBC-based thermoplastic elastomer (TPE), said top panel has less than 20% deformation after 100% stretch for 10 seconds;
one bottom panel in the form of said hand comprising at least one layer made from a second SBC-based thermoplastic elastomer (TPE), said bottom panel has less than 20% deformation after 100% stretch for 10 seconds;
wherein an opening is provided between said top panel and said bottom panel for the insertion of a human hand; the top panel has a first thickness between 0.02 mm and 0.1 mm; and the bottom panel has a second thickness between 0.02 mm and 0.1 mm.

2. A glove as described in claim 1, wherein each of said first and second thermoplastic elastomers comprises a member selected from the group consisting of SIS, SBS, SEBS, SEPS, SEEPS and combinations thereof.

3. A glove as described in claim 1, wherein the Melt Flow Indices of the thermoplastic elastomers are from 0.5 g/10 mins to 10 g/10 mins according to ASTM Test D1238 at 190 C and at a load of 2.16 kg.

4. A glove as described in claim 1, wherein at least one of said thermoplastic elastomers comprises one SIS and a member selected from the group consisting of SBS, SEBS, SEPS and SEEPS.

5. A glove as described in claim 1, wherein at least one of said thermoplastic elastomers comprises one SBS and a member selected from the group consisting of SIS, SEBS, SEPS and SEEPS.

6. A glove as described in claim 1, at least one of the panels is made by a biaxially oriented process.

7. A glove as described in claim 1, wherein the hardness of at least one of the panels is in the range of 35 Shore A to 90 Shore A.

8. A glove as described in claim 1, wherein the hardness of at least one of the panels is in the range of 50 Shore A to 80 Shore A.

9. A glove as described in claim 1, wherein said top panel has less than 10% deformation after 100% stretch for 10 seconds; said bottom panel has less than 10% deformation after 100% stretch for 10 seconds.

10. A disposable glove comprising:
one top panel in the form of a hand with at least one layer made from a first thermoplastic elastomer comprising a first SBC, said first SBC having 12-25% styrene content by weight;
one bottom panel in the form of said hand with at least one layer made from a second thermoplastic elastomer comprising a second SBC, said second SBC having 12-25% styrene content by weight;
wherein a portion of the periphery of said bottom panel is welded to a portion of the periphery of said top panel; an opening is provided between said top panel and said bottom panel for the insertion of a human hand; the top panel has a first thickness between 0.02 mm and 0.1 mm; the bottom panel has a second thickness between 0.02 mm and 0.1 mm.

11. A glove as described in claim 10, where the Melt Flow Indices of the thermoplastic elastomers are from 0.5 g/10 mins to 10 g/10 mins according to ASTM Test D1238 at 190 C and at a load of 2.16 kg.

12. A glove as described in claim 10, wherein each of said first SBC and second SBC is selected from the group consisting of SIS, SBS, SEBS, SEPS, SEEPS and combinations thereof.

13. A glove as described in claim 10, wherein each of said first SBC and second SBC is selected from the group consisting of SIS, SBS, SEBS, SEPS, SEEPS and combinations thereof; and the Melt Flow Indices of the thermoplastic elastomers are from 0.5 g/10 mins to 10 g/10 mins according to ASTM Test D1238 at 190 C and at a load of 2.16 kg.

14. A glove as described in claim 10, wherein each of said thermoplastic elastomers comprises at least two SBC's selected from the group consisting of SIS, SBS, SEBS, SEPS and SEEPS.

15. A glove as described in claim 10, wherein each of said first and second panels has less than 20% deformation after 100% stretch for 10 seconds.

16. A glove as described in claim 10, wherein each of said first and second panel has less than 10% deformation after 100% stretch for 10 seconds.

17. A glove as described in claim 10, wherein at least one of said panels is made by a biaxially oriented process.

18. A glove as described in claim 10, wherein the hardness of one layer of the top or bottom panel is in the range of 50 shore A to 80 Shore A.

19. A disposable glove comprising:
one top panel in the form of a hand with at least one layer made from a first thermoplastic elastomer comprising
a) a first SBC having 12%-25% styrene by weight, and
b) a first polyethylene (PE);
one bottom panel in the form of said hand with at least one layer made from a second thermoplastic elastomer comprising
c) a second SBC having 12%-25% styrene by weight, and
d) a second polyethylene (PE),
wherein a portion of the periphery of said bottom panel is welded to a portion of the periphery of said top panel; an opening is provided between said top panel and said bottom panel for the insertion of a human hand; the top panel has a first thickness between 0.02 mm and 0.1 mm; the bottom panel has a second thickness between 0.02 mm and 0.1 mm; each of said first and second SBC's is selected from the group consisting of SIS, SBS, SEBS, SEPS, and SEEPS.

20. A glove as described in claim 19, wherein the Melt Flow Indices of the thermoplastic elastomers are from 0.5 g/10 mins to 10 g/10 mins according to ASTM Test D1238 at 190 C and at a load of 2.16 kg.

21. A glove as described in claim 19, wherein each of said first and second panels has less than 20% deformation after 100% stretch for 10 seconds.

22. A glove as described in claim 19, wherein each of said first and second panels has less than 10% deformation after 100% stretch for 10 seconds.

23. A glove as described in claim 19, wherein at least one of the panels is made by a biaxially oriented process.

24. A glove as described in claim 19, wherein the hardness of one layer of the top or bottom panel is in the range of 50 Shore A to 80 Shore A.
